(19) United States
(12) Patent Application Publication
(10) Pub. No.: US 2013/0345309 A1
(43) Pub. Date: Dec. 26, 2013

(54) MALIC ACID-ENRICHED PLANT EXTRACT

(71) Applicant: EPC (Beijing) Natural Products Co., Ltd., Beijing (CN)

(72) Inventors: Jingang Shi, Beijing (CN); Wenchao Lu, Beijing (CN)

(73) Assignee: EPC (Beijing) Natural Products Co., Ltd., Beijing (CN)

(21) Appl. No.: 13/800,236

(22) Filed: Mar. 13, 2013

Related U.S. Application Data
(60) Provisional application No. 61/662,424, filed on Jun. 21, 2012.
(60) Provisional application No. 61/776,994, filed on Mar. 12, 2013.

(51) Int. Cl. A23L 1/22; A61K 8/362; A61K 47/12; A61K 31/194; A23L 3/3508; C12P 1/00

(52) U.S. Cl. CPC A23L 1/22066; A23L 3/3508; C12P 1/00; A61K 47/12; A61K 31/194; A61K 8/362
USPC 514/574; 426/654; 426/384; 426/271; 426/650; 426/655; 426/431; 562/580; 435/267; 562/582; 514/784

(57) ABSTRACT

Malic acid-enriched plant extracts useful as food additives are described.

### CROSS REFERENCE TO RELATED APPLICATIONS

This application claims priority to U.S. Provisional Patent Application No. 61/662,424, filed Jun. 21, 2012, and U.S. Provisional Patent Application No. 61/776,994, filed Mar. 12, 2013, both entitled “MALIC ACID-ENRICHED PLANT EXTRACT” which are incorporated by reference in their entirety.

### FIELD OF THE INVENTION

Malic acid-enriched plant extracts useful as food additives are described herein.

### BACKGROUND OF THE INVENTION

Malic acid is an organic compound with the following formula:

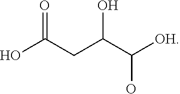

Malic acid has two stereoisomeric forms (L- and D-enantiomers), wherein L-Malic acid is the naturally occurring form, whereas a mixture of L- and D-malic acid is produced synthetically

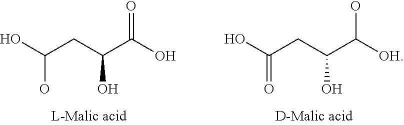 [SMILES: O=C(O)[C@@H](O)CC(O)O ; O=C(O)C[C@@H](O)C(O)O]

Malic acid contributes to the pleasantly sour taste of fruits, and is used as a food additive. The salts and esters of malic acid are known as malates. The malate anion is an intermediate in the citric acid cycle.

Malic acid was first isolated from apple juice by Carl Wilhelm Scheele in 1785. Malic acid contributes to the sourness of green apples. It is present in grapes and in most wines with concentrations sometimes as high as 5 g/l (Methods For Analysis of Musts and Wines”, Ough and Amerine, John Wiley and Sons, 2nd Edition, 1988, page 67). Malic acid occurs naturally in all fruits and many vegetables, and is generated in the fruit metabolic cycle.

Malic acid is regarded as an acid with pleasant tartness, and thus is currently used mainly as an acidulant and taste enhancer in the beverage and food industry. It is also used as a preservative in the food industry. It can be used with or in place of the less sour citric acid in sour sweets. It is approved for use as a food additive in the EU, USA and Australia and New Zealand (where it is listed by its INS number 296). Malic acid, when added to food products, is denoted by E number E296.

Racemic malic acid is synthesized petrochemically from maleic anhydride. The process involves many industrial chemicals, and the product, i.e. DL-malic acid, is not absorbed by humans.

Enantiomerically pure L-malic acid (e.g., for production of foods and pharmaceuticals) is produced from fumarate (synthesized from maleic anhydride) by enantioselective hydration with fumarase, using either immobilized cells or isolated enzymes. Fermentative production of malic acid has been most successfully demonstrated with Aspergillus flavus, achieving 63% of the maximum theoretical yield of malic acid from glucose at high production rates and titers. However, the potential aflatoxin production of the process disqualified A. flavus as a producer of food-grade chemicals (Rintze M. Zelle etc., Malic Acid Production by Saccharomyces cerevisiae: Engineering of Pyruvate Carboxylation, Oxaloacetate Reduction, and Malate Export, APPLIED AND ENVIRONMENTAL MICROBIOLOGY, May 2008, p. 2766-2777).

Malic acid enriches many plants, especially plants of Rosaceae (the rose family). Rosaceae are a medium-sized family of flowering plants, including many species. Among the most important genera are Crataegus (hawthorn), Cotoneaster (cotoneaster), Fragaria (strawberry), Malus (apple), Pyrus (pear), Rosa (rose), Pyracantha (firethorn), Sorbus (rowan), Rubus (blackberry, cloudberry, raspberry), and Prunus (including plums, cherries, peaches, apricots, Japanese apricot, cherry plum, etc.). The most important genus by far is Prunus.

Many plants, especially those in Rosaceae, contain relatively large percentages of malic acid. Chen et al. disclosed that apricots (Prunus armeniaca) contain as much as 45.82 mg/g malic acid (CHEN Mei-xia et al., Relationship between accumulation of organic acid and organic acid-metabolizing enzymes during apricot fruit development, Journal of Fruit Science, 2009, 26(4): 471˜474. Zhai et al. disclosed that Japanese apricot (Prunus mume) contains as much as 0.67 wt. % malic acid (ZHAI Huanchen et al., Simultaneous determination of seven organic acids in Prunus mume with reverse phase high performance liquid chromatography, Fujian Journal of Agricultural Sciences, 22(4): 414˜417, 2007.

Although many plants, especially plants of Rosaceae, contain a large amount of malic acid, their extracts are conventionally prepared as food products, wherein a high content of an organic acid, e.g. malic acid, is generally considered as impairing the overall taste of the food product. Therefore, organic acids are generally removed from such extracts. In addition, many additives, e.g. sweeteners, are generally added in order to improve the flavor and/or taste of the food product that contains the malic acid.

Therefore, a need exists for a malic acid product derived from natural sources, which is free of toxic substances and/or organic solvents, and thus is useful and approvable, e.g. as an acidulant, a taste enhancer, a preservative, etc. that overcomes one or more of the current disadvantages noted above.

### BRIEF SUMMARY OF THE INVENTION

A malic acid-enriched plant extract that has an increased malic acid content as compared to the malic acid content present in naturally occurring native plant materials e.g. 2-100 times, such as about 2, about 5, about 10, about 15, about 20, about 25, about 30, about 50, or about 100 times higher malic acid content, in comparison to (relative to) the amount that occurs naturally in the native plant material is described herein. Suitable sources for plant material include, for example, but not limited to, plants of Rosaceae.

In one aspect, the malic acid-enriched extract is prepared when the fruit is considered immature or “premature”; a time when the malic acid content is high and the fruit is generally not suitable for consumption as the sourness/bitterness would deter an individual from wanting to eat the fruit or drink the juice. Increasing the malic acid content in an extract form would only serve to exacerbate the undesirable sourness/bitterness from the immature fruit and one would not normally seek out such an extract (with an increased concentration of malic acid) until as presented herein.

Generally, the plant of Rosaceae is selected from plants of Crataegus, Cotoneaster, Fragaria, Malus, Pyrus, Rosa, Pyracantha, Sorbus, Rubus and Prunus. More specifically, the plant of Rosaceae is selected from hawthorn (Crataegus pinnatifida), cotoneaster, strawberry (Fragaria ananassa), apple (Malus domestica), pear (Pyrus), rose (Rosa rugosa), firethorn (pyracantha fortuneana), rowan (Sorbus pohuashanensis), blackberry, cloudberry (Rubus chamaemorus), raspberry, plums, cherries, peaches, apricots (Prunus armeniaca), Japanese apricot (Prunus mume), cherry plum (Prunus cerasifera), etc.

In one aspect, the malic acid-enriched plant is obtained from the fruits of plants.

In another aspect, the malic acid-enriched plant is obtained from premature fruits of plants.

The materials provided herein are suitable as food additives, including but not limited to, for various food products. The materials provided herein are also suitable as feeds, nutraceuticals, pharmaceuticals, or cosmetics, for antifatigue ingredients, for exfoliation of the skin, and personal care. In a particular embodiment, one advantage of the materials provided herein is that the processes to arrive at the malic acid-enriched plants described herein do not require the use of toxic substances and/or organic solvent(s) during preparation and isolation.

While multiple embodiments are disclosed, still other embodiments will become apparent to those skilled in the art from the following detailed description. As will be apparent, the descriptions are capable of modifications in various obvious aspects, all without departing from the spirit and scope of the present embodiments described herein. Accordingly, the detailed descriptions are to be regarded as illustrative in nature and not restrictive.

### DETAILED DESCRIPTION

In one aspect, a malic acid-enriched plant extract is described.

In another aspect, a malic acid containing plant juice is used as a preservative, as an acidulant or a taste enhancer for food products, cosmetic products, pharmaceutical agents or personal care. The malic acid containing juice can be obtained from immature or mature plant material, such as plant fruits.

In still another aspect, a malic acid containing plant juice is used as a acidulant or a taste enhancer for food products, cosmetic products, pharmaceutical agents or personal care. The malic acid containing juice can be obtained from immature or mature plant material, such as plant fruits.

The term “juice” as used herein refers to liquid obtained from the crushing, mashing, pulverizing, grinding, or smashing of a plant material, such as the plant fruit. Juice is not concentrated or treated beyond crushing the plant materials to provide the liquid.

As used herein, the phrase “plant extract” means an extract, or concentrate from any part of a plant, such as the seed, leaf, fruit, flower, stem, root, tuber, bark, etc., having an appreciable amount, e.g. higher than about 1%, 2%, 3%, 4%, 5%, etc. through a range of from about 1% to 99% by weight of the total weight of the extract, juice or concentrate, of malic acid. Generally, juice from plants provides less than 4% by weight of malic acid in the juice, more generally less than about 0.5% by weight.

It should be understood that the processes described herein can produce malic acid as the major component of the extract such that a malic acid-enriched extract is provided that comprises substantially 100% malic acid. That is, malic acid can be isolated or purified from the fruit juices or extracts described herein.

In one particular embodiment for the malic acid-enriched extracts described herein, the useful part of the plant suitable for extraction is the fruit.

In a specific embodiment for the malic acid-enriched extracts described herein, the plant is selected from plants of Rosaceae.

In another embodiment for the malic acid-enriched extracts described herein, the plant is selected from plants of Crataegus, Cotoneaster, Fragaria, Malus, Pyrus, Rosa, Pyracantha, Sorbus, Rubus and Prunus.

In still another embodiment for the malic acid-enriched extracts described herein, the plant is selected from hawthorn, cotoneaster, strawberry, apple, pear, rose, firethorn, rowan, blackberry, cloudberry, raspberry, plums, cherries, peaches, apricots, Japanese apricot, cherry plum, etc.

In a particular aspect for the malic acid-enriched extracts described herein, the plant extract is an extract from premature apricots, premature cherry plum and premature Japanese apricots.

The term “enriched” refers to a material, such as an extract, e.g., an apricot containing malic acid extract, that has been subjected to a process to increase amounts of one or more components of the plant relative to the percentages that occur naturally in the plant. For example, the apricot extracts described herein are enriched with malic acid relative to those in the naturally occurring untreated apricot.

As used herein, the term “malic acid-enriched plant extract” means a plant extract that has an increased malic acid content, e.g. 2-100 times, such as about 2, about 5, about 10, about 15, about 20, about 25, about 30, about 50, or about 100 times higher malic acid content, in comparison to (relative to) the amount that occurs naturally in the native plant by weight.

As used herein, the terms “premature” or “immature” refer to plant materials, especially fruits, which have not yet achieved full ripeness. This time period is generally from at least about 1 day to about 1 month, in particular from about 1 day to about 14 days, prior to ripeness and can be determined by those having ordinary skill in the art, such as a produce consumer.

Premature plants, especially fruits of plants, are conventionally much more sourer and bitter than mature fruit (ripened fruit), and thus are considered as distasteful. However, the immature or premature fruit contains increased amounts of organic acids, especially malic acid, oxalic acid, citric acid, etc., and less sugar than matured or ripened fruit. As used herein, the term “ripeness” refers to plant materials, especially fruits, which just begin to change color and can be consumed by a consumer, and can be determined by those having ordinary skill in the art, such as a produce consumer.

In one embodiment, the malic acid-enriched extract and/or the juice can contain one or more flavonoid.

The term “flavonoid(s)” is intended to encompass flavonoids (2-phenylchromen-4-one (2-phenyl-1,4-benzopyrone), isoflavonoids (3-phenylchromen-4-one (3-phenyl-1,4-benzopyrone) and neoflavonoids (4-phenylchromen-4-one (3-phenyl-1,4-benzopyrone). Additionally, flavonoids include flavone glycosides and flavonoid derivatives. Suitable flavanols include rutin and quercitrin.

In an embodiment, the malic acid-enriched plant extract has increased malic acid content higher than that occurs in the native plant. In one embodiment, the malic acid-enriched plant extract has a malic acid content of ≧5 wt. %, particularly ≧10 wt. %, more particularly ≧15 wt. %, such as from about 5 to about 50 wt. %, from about 10 to about 30 wt. %, from about 20 to about 25 wt. %, about 10 wt. %, about 15 wt. %, about 20 wt. %, about 25 wt. %, about 30 wt. %, etc. based on the total weight of the extract.

In another embodiment, the malic acid-enriched plant extract has an increased malic acid content of from about 50 wt. % to about 99 wt. % or higher, in particular from about 60 wt. % to about 95% wt. %, more particularly from about 75 wt. % to about 90 wt. %. In one aspect, the processes described herein would provide a malic acid-enriched extract comprising substantially 100% malic acid.

As discussed above, purified malic acid can be obtained from the processes described herein. At that point, there is only residual plant extract as a possible contaminant providing a malic acid-enriched extract that comprises substantially 100% by weight of malic acid. In one aspect, no residual plant extract remains and malic acid would be the sole product.

The malic acid-enriched plant extract can be extracted from the raw plant materials by any method known in the art, e.g. by extracting with inorganic solvents such as water and/or organic solvents, including alcohols such as methanol, ethanol, butanol, propanol, isopropanol, etc.; ethers such as ethyl ether, tetrahydrofuran, etc.; esters such as ethyl acetate; aromatic hydrocarbons such as benzene, toluene, and derivatives thereof and other organic solvents such as dichloromethane and chloroform; and combinations thereof.

For example, a malic acid-enriched plant extract can be prepared by a method comprising the steps of: 1) mixing the plant material with inorganic solvent(s) and/or organic solvent(s), optionally with stirring; 2) heating the mixture to a temperature of room temperature to reflux, and then cooling the mixture to room temperature; 3) filtering the mixture and washing the filter cake with the solvent(s); 4) repeating the steps of 1) through 3) at least once, 5) combining the filtrates of the filtration processes and concentrating the combined filtrates at a temperature of room temperature to the boiling temperature of the solvent(s), to obtain the final malic acid-enriched plant.

In one particular aspect, the malic acid-enriched plant extract does not contain any appreciable (detectable) amounts of toxic substances and/or organic solvents, and thus is useful and approvable, e.g. as a food additive such as acidulant, taste enhancer, preservative, etc., in the production of food products, especially “green foods”.

In an aspect, a method for preparing the malic acid-enriched plant extract is provided.

For example, a malic acid-enriched plant extract can be prepared by a method comprising the steps of: 1) mixing the plant material with water, optionally with stirring, wherein the weight ratio of plant material to water is in the range of from about 0.5 to about 20, from about 1 to about 10, more particularly from about 2 to about 5, such as 1.0, 2.0, 3.0, 5.0, etc., to form a mixture; 2) heating the mixture to a temperature of 40° C. to reflux, e.g. 60° C. or 70° C., for e.g., about 0.5 hour to about 20 hours, and then cooling the mixture to room temperature; 3) filtering the mixture and washing the filter cake with water; 4) repeating the steps of 1) through 3) at least once, twice, three times, etc., 5) combining the filtrates of the filtration processes and concentrating the combined filtrates at a temperature of room temperature to about 100° C., e.g. 50° C., 60° C., 65° C., 70° C., etc., to obtain the final malic acid-enriched plant.

In one aspect, the plant material can obtained from a fresh plant. In another aspect, the plant material can be processed before being used, for example, the plant material can be frozen, dried, or can be stored in a warm environment for several days before being used.

The process described herein does not involve the use of any chemical substance(s) to extract the malic acid except for water, and thus the malic acid-enriched plant extract prepared according to methods described herein does not contain any toxic substance(s) and/or organic solvent(s).

In another aspect, the use of the malic acid-enriched plant extracts described herein can be used as food additives, especially as an acidulant, a taste enhancer, or as a preservative, in the manufacture of food products, especially “green foods”.

The malic acid-enriched plants described herein can be incorporated into various foods, drinks, snacks, etc. In one aspect, the malic acid-enriched plant can be added to a food product, prior to consumption. If added to a food product, a suitable carrier such as starch, sucrose or lactose, can be used to help distribute the concentration of the malic acid-enriched material making it easier to apply to the food product.

The malic acid-enriched plants described herein can also be provided as supplements in various prepared food products. For the purpose of this application, prepared food product means any natural, processed, diet or non-diet food product to which an malic acid-enriched plant described herein has been added. The malic acid-enriched plants described herein can be directly incorporated into many prepared diet food products, including, but not limited to diet drinks, diet bars and prepared frozen meals. Furthermore, the malic acid-enriched plants described herein can be incorporated into many prepared non-diet products, including, but not limited to candy, snack products such as chips, prepared meat products, milk, cheese, yogurt, sport bars, sport drinks, mayonnaise, salad dressing, bread and any other fat or oil containing foods. As used herein, the term “food product” refers to any substance fit for human or animal consumption.

The malic acid-enriched plants described herein can be added to various drinks, such as fruit juices, milkshakes, milk, etc.

Furthermore, the malic acid-enriched extracts described herein can be mixed with other plant extracts e.g. extracts from bitter melon, mulberry leaves, stevia, etc.

In the specification and in the claims, the terms “including” and “comprising” are open-ended terms and should be interpreted to mean “including, but not limited to . . . ” These terms encompass the more restrictive terms “consisting essentially of” and “consisting of”.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this technology belongs. All publications and patents specifically mentioned herein are incorporated by reference in their entirety for all purposes including describing and disclosing the chemicals, instruments, statistical analyses and methodologies which are reported in the publications which might be used in connection with the embodiments described herein. All references cited in this specification are to be taken as indicative of the level of skill in the art. Nothing herein is to be construed as an admission that the embodiments are not entitled to antedate such disclosure by virtue of prior invention.

The following paragraphs enumerated consecutively from 1 through 64 provide for various aspects of the subject matter described herein. In one embodiment, in a first paragraph (1), a malic acid-enriched plant extract, comprising a plant extract having an increased malic acid content relative to the malic acid content that occurs naturally in the native plant at a specified time is provided.

2. The malic acid-enriched plant extract according to paragraph 1, wherein the malic acid-enriched extract has an increased malic acid content of from about 2 to about 100 times in comparison to the amount that is present in the native plant.

3. The malic acid-enriched plant extract according to paragraph 2, wherein the malic acid-enriched extract has about 2, about 5, about 10, about 15, about 20, about 25, about 40, about 50, or about 100 times increased malic acid content, in comparison to the amount that is present in the native plant.

4. The malic acid-enriched plant extract according to paragraph 1, wherein the malic acid-enriched extract has a malic acid content of ≧5 wt. % based on the total weight of the extract.

5. The malic acid-enriched plant extract according to paragraph 4, wherein the malic acid-enriched extract has a malic acid content of ≧10 wt. % based on the total weight of the extract.

6. The malic acid-enriched plant extract according to paragraph 5, wherein the malic acid-enriched extract has a malic acid content of ≧15 wt. % based on the total weight of the extract.

7. The malic acid-enriched plant extract according to paragraph 4, wherein the malic acid-enriched extract has a malic acid content of about 5 to about 50 wt. % based on the total weight of the extract.

8. The malic acid-enriched plant extract according to paragraph 7, wherein the malic acid-enriched extract has a malic acid content of about 10 to about 30 wt. % based on the total weight of the extract.

9. The malic acid-enriched plant extract according to paragraph 8, wherein the malic acid-enriched extract has a malic acid content of about 20 to about 25 wt. % based on the total weight of the extract.

10. The malic acid-enriched plant extract according to paragraph 4, wherein the malic acid-enriched extract has a malic acid content of about 10 wt. %, about 15 wt. %, about 20 wt. %, about 25 wt. %, or about 30 wt. % based on the total weight of the extract.

11. The malic acid-enriched plant extract according to any one of paragraphs 1-10, wherein the plant material is selected from the plants of Rosaceae.

12. The malic acid-enriched plant extract according to paragraph 11, wherein the plant material is selected from the plants of Crataegus, Cotoneaster, Fragaria, Malus, Pyrus, Rosa, Pyracantha, Sorbus, Rubus, Prunus or mixtures thereof.

13. The malic acid-enriched plant extract according to paragraph 12, wherein the plant material is selected from hawthorn, cotoneaster, strawberry, apple, pear, rose, firethorn, rowan, blackberry, cloudberry, raspberry, plums, cherries, peaches, apricots, Japanese apricot, cherry plum or mixtures thereof.

14. The malic acid-enriched-enriched plant extract according to any one of paragraphs 1 through 13, wherein the extract is obtained from fruit of the plant.

15. The malic acid-enriched-enriched plant extract according to any one of paragraphs 1 through 14, wherein the extract is obtained from immature fruit of the plant.

16. The malic acid-enriched plant extract according to any of paragraphs 1 through 15, wherein the plant is a fresh plant.

17. The malic acid-enriched plant extract according to any one of paragraphs 1 through 16, wherein the plant has been processed before being used.

18. The malic acid-enriched plant extract according to any of paragraphs 1 through 17, wherein the plant extract is free of toxic substance(s) and organic solvent(s).

19. A process for preparing the malic acid-enriched plant extract according to any one of paragraphs 1 through 18, comprising the steps of:

1) mixing the plant material with water, optionally with stirring, to form a mixture;

2) heating the mixture to a temperature of about 40° C. to reflux and then cooling the mixture to room temperature;

3) filtering the mixture and washing the filter cake with water;

4) performing the steps of 1) through 3) at least once, and

5) combining the filtrates of the filtration processes and concentrating the combined filtrates at a temperature of room temperature to 100° C., to obtain the final extract.

20. The process according to paragraph 19, wherein the weight ratio of plant material to water in step 1) is in the range of from about 0.5 to about 20.

21. The process according to paragraph 20, wherein the weight ratio of plant material to water in step 1) is in the range of from about 1 to about 10.

22. The process according to paragraph 21, wherein the weight ratio of plant material to water in step 1) is in the range of from about 2 to about 5.

23. The process according to paragraph 19, wherein the weight ratio of plant material to water in step 1) is in the range of 1.0, 2.0, 3.0, or 5.0.

24. The process according to paragraph 19, wherein the mixture is heated to 60° C. or 70° C. in step 2).

25. The process according to paragraph 19, wherein the mixture is heated for about 0.5 hour to about 20 hours.

26. The process according to paragraph 19, wherein the steps of 1) through 3) are repeated once, twice or three times.

27. The process according to paragraph 19, wherein the combined filtrate is concentrated at a temperature of 50° C., 60° C., 65° C., or 70° C. in step 5).

28. Use of the malic acid-enriched plant extract according to any of paragraphs 1 through 27 in the food industry.

29. The use according to paragraph 28, wherein the malic acid-enriched plant extract is used as an acidulant, a taste enhancer, or a preservative.

30. Use of the malic acid-enriched plant extract according to any of paragraphs 1 through 27 in feeds, nutraceuticals, pharmaceuticals, or cosmetics.

31. The use according to paragraph 30, wherein the malic acid-enriched plant extract is used for antifatigue ingredient, exfoliation of the skin, and/or personal care.

32. A method to preserve a food product, a pharmaceutical agent or personal care products, comprising the step of adding plant juice obtained from an immature plant material having malic acid, selected from the plants of Rosaceae.

33. The method of paragraph 32, wherein the immature plant material is selected from the plants of Crataegus, Cotoneaster, Fragaria, Malus, Pyrus, Rosa, Pyracantha, Sorbus, Rubus, Prunus or mixtures thereof.

34. The method of either of paragraphs 32 or 33, wherein the plant juice is obtained from fruit of the plant.

35. A method to preserve a food product, a pharmaceutical agent or personal care products, comprising the step of adding plant juice obtained from a plant material having malic acid, selected from the plants of Rosaceae.

36. The method of paragraph 35, wherein the plant material is selected from the plants of Crataegus, Cotoneaster, Fragaria, Malus, Pyrus, Rosa, Pyracantha, Sorbus, Rubus, Prunus or mixtures thereof.

37. The method of either of paragraphs 35 or 36, wherein the plant juice is obtained from fruit of the plant.

38. The method of any of paragraphs 32 through 37, wherein the plant juice is free of toxic substance(s) and organic solvent(s).

39. A method to increase the acidulant effect in a food product, in a pharmaceutical agent or in personal care products, comprising the step of adding a suitable amount of plant juice obtained from an immature plant material having malic acid, selected from the plants of Rosaceae to increase the acidulant taste of the product.

40. The method of paragraph 39, wherein the immature plant material is selected from the plants of Crataegus, Cotoneaster, Fragaria, Malus, Pyrus, Rosa, Pyracantha, Sorbus, Rubus, Prunus or mixtures thereof.

41. The method of either of paragraphs 39 or 40, wherein the plant juice is obtained from fruit of the plant.

42. A method to increase the acidulant effect in a food product, in a pharmaceutical agent or in personal care products, comprising the step of adding plant juice obtained from a plant material having malic acid, selected from the plants of Rosaceae to increase the acidulant taste of the product.

43. The method of paragraph 42, wherein the plant material is selected from the plants of Crataegus, Cotoneaster, Fragaria, Malus, Pyrus, Rosa, Pyracantha, Sorbus, Rubus, Prunus or mixtures thereof.

44. The method of either of paragraphs 42 or 43, wherein the plant juice is obtained from fruit of the plant.

45. The method of any of paragraphs 39 through 44, wherein the plant juice is free of toxic substance(s) and organic solvent(s).

46. A method to enhance taste in a food product, in a pharmaceutical agent or in personal care products, comprising the step of adding a suitable amount of plant juice obtained from an immature plant material having malic acid, selected from the plants of Rosaceae to enhance the taste of the product.

47. The method of paragraph 46, wherein the immature plant material is selected from the plants of Crataegus, Cotoneaster, Fragaria, Malus, Pyrus, Rosa, Pyracantha, Sorbus, Rubus, Prunus or mixtures thereof.

48. The method of either of paragraphs 46 or 47, wherein the plant juice is obtained from fruit of the plant.

49. A method to enhance taste in a food product, in a pharmaceutical agent or in personal care products, comprising the step of adding plant juice obtained from a plant material having malic acid, selected from the plants of Rosaceae to enhance the taste of the product.

50. The method of paragraph 49, wherein the plant material is selected from the plants of Crataegus, Cotoneaster, Fragaria, Malus, Pyrus, Rosa, Pyracantha, Sorbus, Rubus, Prunus or mixtures thereof.

51. The method of either of paragraphs 49 or 50, wherein the plant juice is obtained from fruit of the plant.

52. The method of any of paragraphs 46 through 51, wherein the plant juice is free of toxic substance(s) and organic solvent(s).

53. A process for preparing the malic acid-enriched plant extract according to any one of paragraphs 1 through 17, comprising the steps of:

1) mixing the plant material with inorganic solvent(s) except water and/or organic solvent(s), optionally with stirring, to form a mixture;

2) heating the mixture to a temperature of room temperature to reflux and then cooling the mixture to room temperature;

3) filtering the mixture and washing the filter cake with the solvent(s);

4) performing the steps of 1) through 3) at least once, and

5) combining the filtrates of the filtration processes and concentrating the combined filtrates at a temperature of room temperature to the boiling temperature of the solvent(s), to obtain the final extract.

54. The process according to paragraph 53, wherein the inorganic solvent is selected from inorganic acids, carbon disulfide, and carbon dioxide.

55. The process according to paragraph 53, wherein the organic solvent is selected from alcohols, ethers, esters, and aromatic hydrocarbons.

56. The process according to paragraph 55, wherein the organic solvent is selected from methanol, ethanol, butanol, propanol, isopropanol; ethyl ether, tetrahydrofuran; ethyl acetate; benzene, toluene, and derivatives thereof; dichloromethane, and chloroform, and combinations thereof.

57. The malic acid-enriched plant extract according to any of paragraphs 1 through 18, further comprising one or more flavonoids.

58. A process for preparing the malic acid-enriched plant extract according to any one of paragraphs 1-18, comprising the steps of:

1) mixing a plant material with water, optionally with stirring, to form a mixture;

2) filtering the mixture and washing the filter cake with water;

3) performing the steps of 1)-2) at least once,

4) combining the filtrates of the filtration processes and optionally treating the combined filtrate with pectinase for a period of time from about 5 minutes to about 24 hours, e.g., overnight, over a temperature range of from about 0° C. to about 70° C., in particular from about 40° C. to about 60° C.; and

purifying the resultant mixture, such as by adsorption, decolorization with activated carbon, silica gel, alumina, molecular sievse, white clay, diatomite, zeolite, centrifugation, extraction, distillation, crystallization, ion-exchange, precipitation, freeze drying, filtration, column chromatography or combinations thereof, to provide a malic acid-enriched plant extract material.

59. The process according to paragraph 58, wherein the filtration step is by membrane filtration, ultrafiltration or reverse osmosis.

60. The process according paragraph 58, wherein at least one purification step is by membrane filtration, ultrafiltration, or reverse osmosis, adsorption, column chromatography, centrifugation, extraction, distillation, crystallization, ion-exchange, precipitation, or freeze drying.

61. The process according to any of paragraphs 58 through 60, wherein a decolorization process is performed after the purification process.

62. The process according to any of paragraphs 58 through 61, further comprising the steps:

combining the malic acid-enriched plant extract material with a carrier, such as cellulose derivatives, starch derivatives, dextrin derivatives, cyclodextrins, maltodextrin, agar, or combinations thereof, to form a mixture; and

subjecting the mixture to spray drying conditions to provide a powdered solid.

63. The process according to any of paragraphs 19-27 or 53 through 62, wherein the malic acid-enriched extract comprises substantially 100% malic acid.

64. A malic acid-enriched extract comprising substantially 100% malic acid.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

### EXAMPLES

1. Reagents

Methanol: HPLC grade, B&J ACS, USA

Purified Water: Wahaha purified water, Hangzhou, Zhejiang, China

Citric acid: analytical reagent, Beijing Chemical Works, China

L-Malic acid: analytical reagent, Beijing Chemical Reagent Plant, China

KH₂PO₄: analytical reagent, Tianjin Guangfu Technology Development Co. Ltd., China
2. Instrument and chromatographic conditions

Instrument: Agilent 1100 LC system (Agilent Technologies, Palo Alto, Calif.)

Column: Agilent ZORBAX SB-C₁₈ (4.6×250 mm, 5 μm)

Wavelength: 210 nm

Temperature: room temperature

Flow: 0.8 ml/min

Inject volume: 10 μl

Detection Systems:VWD

Mobile Phase: A: methanol, B: 0.01 mol/l KH₂PO₄ buffer solution (pH=2.85)

| | A | B |
|---|---|---|
| 0 min | 0% | 100% |
| 10 min | 0% | 100% |
| 15 min | 45% | 55% |

| | |
|---|---|
| 25 min | stop |
| 7 min | post time |

3. Test Solutions

Preparation of Standard Solutions:

15 mg L-malic acid was put into a 5 ml volumetric flask, dissolved with purified water to the mark, and filtered through a 0.22 μm membrane.

15 mg citric acid was put into a 50 ml volumetric flask, dissolved with purified water to the mark, and filtered through a 0.22 μm membrane.

Preparation of Test Samples:

200 mg test sample was put into a 10 ml volumetric flask, dissolved with purified water to the mark, and filtered through a 0.22 μm membrane.
4. Calculation

$$Content \% = \frac{{A}_{test} \times {W}_{reference} \times {V}_{test}}{{A}_{reference} \times {W}_{test} \times {V}_{reference}} \times 100 \%$$

Aₜₑₛₜ: the peak area of test samples

A_{reference}: the peak area of reference samples

W_{reference}: weight of reference samples (mg)

Wₜₑₛₜ: weight of test samples (mg)

Vₜₑₛₜ: dilution factor of test samples (ml)

V_{reference}: dilution factor of reference samples (ml)

### Example 1

329 g of green (premature) apricot was crushed and the kernel was removed. The crushed apricot was then mixed with 800 ml water to obtain a first mixture. The first mixture was stirred at 70° C. for 2 hours, cooled to room temperature, and then was filtered to obtain a first filter cake. The first filter cake was washed with 100 ml water, and then was mixed with 800 ml water to obtain a second mixture. The second mixture was stirred at 70° C. for 2 hours, cooled to room temperature, and then was filtered to obtain a second filter cake. The second filter cake was washed with 100 ml water, and then was mixed with 800 ml water to obtain a third mixture. The third mixture was stirred at 70° C. for 2 hours, cooled to room temperature, and then filtered to obtain a third filter cake. The third filter cake was washed with 100 ml water. The filtrates obtained from the three filtration processes were combined and were concentrated at 65° C. under reduced pressure, to obtain 35 g of an extract in the form of a yellow sticky liquid with a malic acid content of 20% and a citric acid content of 1.6%, as determined by HPLC. Percentage is weight percentage.

### Example 2

500 g of Prunus mume var. bungo Makino fruit (from Fucheng, China) was crushed and the kernel was removed. The crushed fruit was then mixed with 1000 ml deionized water, the resultant mixture was stirred for 2 hours and was filtered to provide a first filtrate. The residue was mixed with 1000 ml deionized water to form a mixture, the mixture was stirred for 2 hours, and was filtered to provide a second filtrate. The residue was mixed with 500 ml deionized water to form a mixture, the mixture was stirred for 2 hours and was filtered to provide a third filtrate, and the residue was discarded. The 3 filtrates from the 3 filtration processes were combined to obtain 2500 g filtrate. 0.25 g pectinase (from Imperial Jade Bio-technology Co., Ltd) was added to the combined filtrate, the combined filtrate was held at 50° C. for 4 hours and was then was filtered through a membrane with molecular weight cutoff of 10000 Da in a membrane filtration device (from Suntar Membrane Technology (Xiamen) Co., Ltd). The filtrate from the membrane filtration separation was loaded onto a column filled with 250 g AB-8 resin (from The Chemical Plant of NanKai University) for decolorization. A part of the decolorized filtrate was concentrated at 65° C. to obtain 50 g product in the form of yellow sticky liquid. The malic acid content of the yellow sticky liquid was determined to be 21.38%.

The yellow sticky liquid could be neutralized with calcium fumarate to pH 6.9 to 7.2, and the system could be stirred for 30 minutes and then the filter cake would be filtered and washed with 100 ml water.

The filter cake that would be obtained from above would be treated with 150 ml water in a 400 ml flask, then sulfuric acid would be added to adjust pH to 1.5 to 2.0. The system would then be concentrated under reduced pressure at 65° C. to about 40 gram, cooled to 15° C., and then centrifuged to obtain 20 gram product as a white solid. The white solid would be vacuum dried to obtain 12 gram white solid with L-malic acid content of 95%.

40 g maltodextrin (about 4 equivalent compared to the malic acid in the liquid) was added into the yellow sticky liquid, the mixture was dissolved in water at room temperature, and then was spray dried (inlet temperature=120° C., outlet temperature=85° C.) to obtain 60 g product in the form of pale yellow solid (powder).

| The components of the yellow sticky liquid and | Malic acid | Citric acid |
|---|---|---|
| the pale yellow solid were analyzed | content | content |
| yellow sticky liquid | 21.38% | 2.37% |
| pale yellow solid | 15.32% | 1.29% |

Malic acid and citric acid content were determined by HPLC analysis as noted above.

The inventions illustratively disclosed herein suitably may be practiced in the absence of any element which is not specifically disclosed herein. Additionally, the inventions illustratively disclosed herein may be practiced in the absence of any element disclosed herein.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A malic acid-enriched plant extract, comprising a plant extract having an increased malic acid content relative to the malic acid content that occurs naturally in the native plant at a specified time.

2. The malic acid-enriched plant extract according to claim 1,
wherein the malic acid-enriched extract has a malic acid content of ≧20 wt. % based on the total weight of the extract.

3. The malic acid-enriched plant extract according to claim 1,
wherein the malic acid-enriched extract has a malic acid content of about 20 to about 25 wt. % based on the total weight of the extract.

4. The malic acid-enriched plant extract according to claim 1, wherein the plant material is selected from the plants of Rosaceae.

5. The malic acid-enriched plant extract according to claim 1, wherein the plant material is selected from the plants of Crataegus, Cotoneaster, Fragaria, Malus, Pyrus, Rosa, Pyracantha, Sorbus, Rubus, Prunus or mixtures thereof.

6. The malic acid-enriched plant extract according to claim 1, wherein the plant extract is free of toxic substance(s) and organic solvent(s).

7. A process for preparing the malic acid-enriched plant extract, comprising the steps of:
1) mixing a plant material with water, optionally with stirring, to form a mixture;
2) filtering the mixture and washing the filter cake with water;
3) performing the steps of 1)-2) at least once,
4) combining the filtrates of the filtration processes and optionally treating the combined filtrate with pectinase for a period of time from about 5 minutes to about 24 hours over a temperature range of from about 0° C. to about 70° C.; and
5) purifying the resultant mixture, to provide a malic acid-enriched plant extract material.

8. The process of claim 7, wherein purifying comprises adsorption.

9. The process of claim 8, wherein adsorption is with activated carbon, silica gel, alumina, molecular sieves, white clay, diatomite, zeolites, or mixtures thereof.

10. The process of claim 7, wherein purifying comprises centrifugation, extraction, distillation, crystallization, ion-exchange, precipitation, freeze drying, filtration, column chromatography or combinations thereof.

11. The process according to claim 7, wherein the filtration step is by membrane filtration, ultrafiltration or reverse osmosis.

12. The process according to claim 7, wherein a decolorization process is performed after the purification process.

13. The process according to claim 7, further comprising the steps:
combining the malic acid-enriched plant extract material with a carrier, to form a mixture; and
subjecting the mixture to spray drying conditions to provide a powdered solid.

14. The process according to claim 13, wherein the carrier is one of cellulose derivatives, starch derivatives, dextrin derivatives, cyclodextrins, maltodextrin, agar, or combinations thereof.

15. The process according claim 9, wherein the malic acid-enriched extract comprises substantially 100% malic acid.

16. A malic acid-enriched plant extract comprising substantially 100% malic acid.

17. The process according to claim 7, wherein the extract is obtained from fruit of the plant.

18. The process according to claim 17, wherein the extract is obtained from immature fruit of the plant.

19. Use of the malic acid-enriched plant extract according to claim 1 in the food industry.

20. The use according to claim 19, wherein the malic acid-enriched plant extract is used as an acidulant, a taste enhancer, or a preservative.

21. Use of the malic acid-enriched plant extract according to claim 1 in feeds, nutraceuticals, pharmaceuticals, or cosmetics.

22. The use according to claim 21, wherein the malic acid-enriched plant extract is used for antifatigue ingredient, exfoliation of the skin, and/or personal care.

23. A method to preserve a food product, a pharmaceutical agent or personal care products, comprising the step of adding plant juice obtained from an immature plant material having malic acid, selected from the plants of Rosaceae.

24. A method to increase the acidulant effect in a food product, in a pharmaceutical agent or in personal care products, comprising the step of adding a suitable amount of plant juice obtained from an immature plant material having malic acid, selected from the plants of Rosaceae to increase the acidulant taste of the product.

25. A method to enhance taste in a food product, in a pharmaceutical agent or in personal care products, comprising the step of adding a suitable amount of plant juice obtained from an immature plant material having malic acid, selected from the plants of Rosaceae to enhance the taste of the product.
